(19) United States
(12) Patent Application Publication
(10) Pub. No.: US 2006/0084807 A1
(43) Pub. Date: Apr. 20, 2006

(54) Novel denzamide derivatives and process for production thereof

(76) Inventors: Shinya Yoshida, Takahagi-shi (JP); Toshihiro Watanabe, Tsukuba-shi (JP); Kiyotaka Marumo, Takahagi-shi (JP); Akio Kakefuda, Tsukuba-shi (JP)

Correspondence Address: SUGHRUE MION, PLLC, 2100 PENNSYLVANIA AVENUE, N.W., SUITE 800, WASHINGTON, DC, 20037, US

(21) Appl. No.: 10/540,421

(22) PCT Filed: Dec. 24, 2003
(86) PCT No.: PCT/JP03/16582

§ 371 Date: Jun. 24, 2005

(30) Foreign Application Priority Data
Dec. 25, 2002 (JP) 2002-375153

(51) Int. Cl. C07D 401/02

(52) U.S. Cl. 546/146

(57) ABSTRACT

There are provided a novel process for the production of an isoquinoline derivative having an inhibitory action for I_{f} current; a novel benzamide derivative or a salt thereof used for the production process; and a process for the production of the benzamide derivative or a salt thereof.

### TECHNICAL FIELD

The present invention relates to a novel benzamide which is a useful intermediate for the manufacture of an isoquinoline derivative useful as an I_{f} current inhibitor, to a process for producing the same and to a process for producing the isoquinoline derivative using the novel benzamide derivative.

### BACKGROUND ART

It has been known that the isoquinoline derivative represented by the following formula (A) has an inhibitory action for I_{f} current and shows a strong and specific activity selectively lowering a heart beat and decreasing oxygen consumption of heart muscle whereby it is useful as a preventive and/or treating agent for diseases of circulatory system such as ischemic heart diseases (e.g., angina pectoris and myocardial infarction), congestive heart failure, arrhythmia, etc. (Patent Document 1).

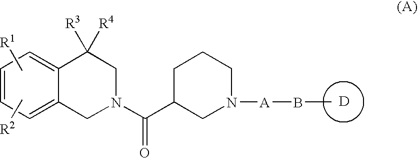

(Refer to the Corresponding Patent Gazette for Symbols in the Formula.)

Among the above-mentioned isoquinoline derivatives, it is mentioned in Examples of the patent gazette that the particular compound where A is ethylene, B is —NH—(C(═O)—, both R³ and R⁴ are hydrogen and a ring D is an optionally substituted phenyl was able to be produced according to the following process for the production (hereinafter, referred to “production process X”). 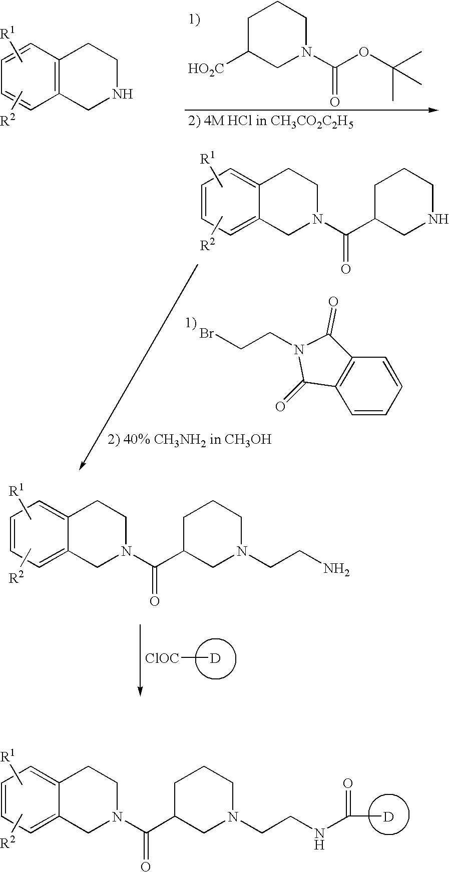
(Refer to the Corresponding Patent Gazette for Symbols in the Formula.)

However, according to the description of the patent gazette, the overall yield for producing N-{2-[3-(6,7-dimethoxy-1,2,3,4-tetrahydroisoquinoline-2-carbonyl)piperidino]ethyl}-3,4-methylenedioxybenzamide hydrochloride which is an aimed compound from 6,7-dimethoxy-1,2,3,4-tetrahydroisoquinoline is about 16% (refer to Referential Example 2, Referential Example 3 and Example 9 of the patent gazette) and the efficiency is very poor. In addition, purification using column chromatography is necessary and it is not preferred as an industrial process for production. Further, in benzoylation of a primary amine which is the final step of the production process X, a diacyl compound may be produced and it is greatly presumed that control of the reaction condition is difficult. Further, an amino derivative which is protected as phthalimide is used and, therefore, a waste product is resulted by deprotection and there is a problem as an industrial production process. The use of chloroform in an extracting step and in a purifying step in column chromatography is not favorable in view of environment as well.

Under such circumstances, there has been a brisk demand for the development of an excellent production process for an isoquinoline derivative with a higher overall yield by column chromatography where purification is not necessary and productivity in view of industrial production is high.

[Patent Document 1] WO 00/75133

### DISCLOSURE OF THE INVENTION

The present inventors have carried out intensive investigations for another production process for an isoquinoline derivative and, as a result, the inventors have found that a novel production process as shown in the following production process 2-1 and production process 2-2 is an excellent process for the production of isoquinoline derivative and that a novel benzarnide derivative represented by the following formula (I) used as a material in the production process as such is an excellent intermediate for a synthesis of the isoquinoline derivative in a high yield whereupon the invention has been achieved.
(Production Process 2-1) 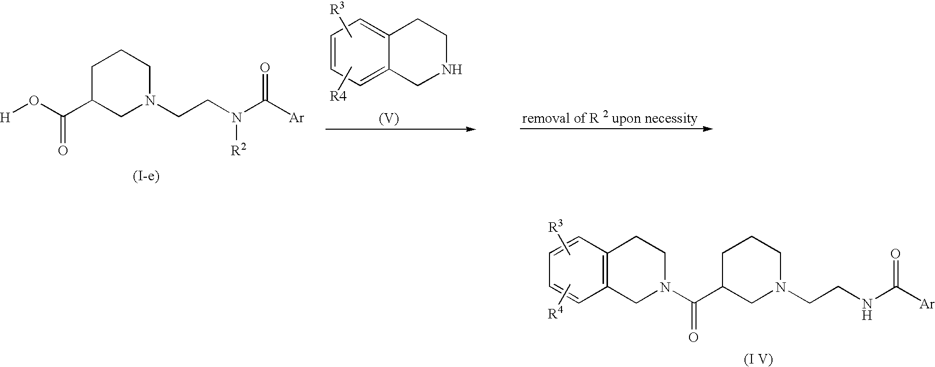
(Symbols in the Formulae will be Illustrated Later.)
(Production Process 2-2)

Removal of R² upon necessity 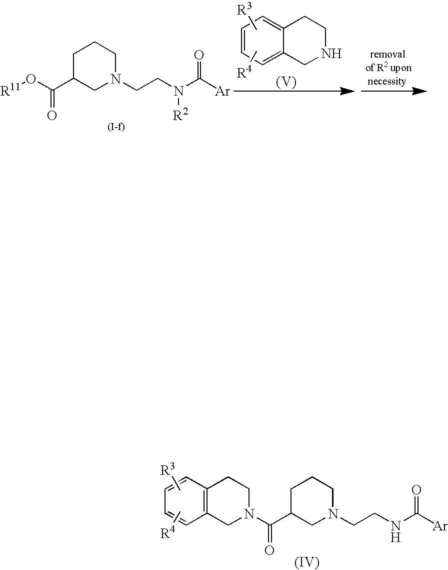
(Symbols in the Formulae will be Illustrated Later.)

Thus, in accordance with the invention, there is provided a novel benzamide derivative represented by the following formula (I) or a salt thereof which is useful as an intermediate for a novel process for the production of an isoquinoline derivative represented by the formula (IV). 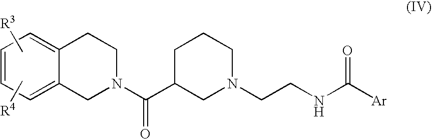
(In the formula, R³and R⁴ may be same or different and each is —H, lower alkyl or —O-lower alkyl; and Ar is aryl which may be substituted. Hereinafter, they have the same meanings as well.) 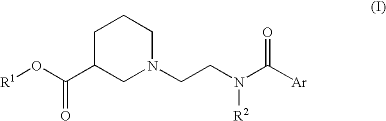
(In the formula, R¹ is —H or an ester residue; and R² is —H or a protective group for amino group. Hereinafter, they have the same meanings as well.)

Further, in accordance with the invention, there is provided a process for the production of a novel benzamide derivative represented by the formula (I) or a salt thereof in which R² is —H comprising a reaction in which a dihydrooxazole derivative represented by the formula (II) 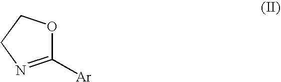
with a nipecotic acid derivative represented by the formula (III) 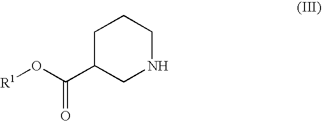
under an acidic condition and, when R¹ is a group other than —H, R¹ is removed if necessary.

Still further, in accordance with the invention, there is provided a process for the production of the isoquinoline derivative represented by the above formula (IV), wherein, when the compound represented by the formula (I) is subjected, if necessary, to a reaction for removing R¹ and/or R² when R¹ and/or R² are/is group(s) other than —H, then condensed with a tetrahydroisoquinoline derivative represented by the formula (V) or a salt thereof and, when R² is a group other than —H, it is subjected to a reaction for removal of R².

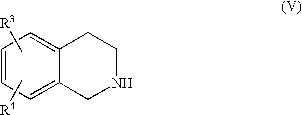

The invention will now be further illustrated as follows.

In the present specification, “lower-alkyl” is a C₁₋₆ straight or branched alkyl and, to be more specific, it is methyl, ethyl, propyl, butyl, pentyl or hexyl or structural isomer thereof such as isopropyl and, preferably, it is a C₁₋₄ alkyl or, more preferably, methyl or ethyl.

“Aryl” is a monovalent group of a monocyclic to tricyclic C₆₋₁₄ aromatic hydrocarbon ring. To be more specific, it is phenyl, naphthyl, etc. and, preferably, it is phenyl.

With regard to “ester residue” in R¹, any group may be used so far as it is a group which is able to be converted to HO—(C+O)— by a reaction for removal of R¹ from R¹O—(C═O)—. To be more specific, groups mentioned in “Protective Groups in Organic Synthesis” (Third Edition) by Greene and Wuts may be exemplified. Preferred examples are lower alkyl and benzyl, more preferred examples are methyl, ethyl and tert-butyl and a particularly preferred example is ethyl.

With regard to R³ and R⁴, —O-lower alkyl is preferred, methoxy is more preferred and the case where R³ is 6-methoxy and R⁴ is 7-methoxy or, in other words, 6,7-dimethoxy-1,2,3,4-tetrahydroisoquinoline as the compound (V) is still more preferred.

With regard to a group allowed in “an optionally substituted aryl” for Ar, any group may be used so far as it is usually able to be substituted to aryl and specific examples are halogen, lower alkyl and —O-lower alkyl. An optionally substituted aryl represented by Ar may be one or more and same or different group(s) as such. With regard to Ar, phenyl substituted with one or more halogen is preferred; phenyl substituted with one halogen is more preferred; phenyl substituted with one halogen at 4-position is still more preferred; and 4-fluorophenyl is particularly preferred.

With regard to “protective group for amino group” for R², any group may be used so far as it is a substituent on nitrogen being able to be removed without affecting other functional group of the compound represented by the formula (I) or (IV) and specific examples are the groups mentioned in the above-mentioned “Protective Groups in Organic Synthesis” (Third Edition). To be more specific, tert-butoxycarbonyl group, methoxy methyl group, tert-butyidimethylsilyl group, etc. may be exemplified. With regard to R², —H is preferred.

With regard to “reaction for removal of R¹”, any reaction may be used so far as it is a reaction which is able to remove R¹ without affecting other functional groups in the compound represented by the formula (I) and its specific examples are hydrolysis with an alkali, hydrolysis with an acid and a catalytic reduction reaction.

With regard to “reaction for removal of R²”, any reaction may be used so far as it is a reaction which is able to remove R² without affecting other functional groups in the compound represented by the formula (I) or (IV). Its specific examples are the reactions mentioned in the above-mentioned “Protective Groups in Organic Synthesis” (Third Edition) and, to be more specific, a method using a reagent such as trimethylsilyl trifluoromethane sulfonate, boron tribromide, etc., hydrolysis using an acid and the like may be exemplified.

In the present specification, the compounds represented by the formulae (I), (III) and (IV) have one or more asymmetric carbon and, therefore, there are optical isomers. The invention includes a mixture of such optical isomers or an isolated one thereof and a process for production using the same. Among those optical isomers, preferred one is an (R)-substance or the compounds represented by the formula.

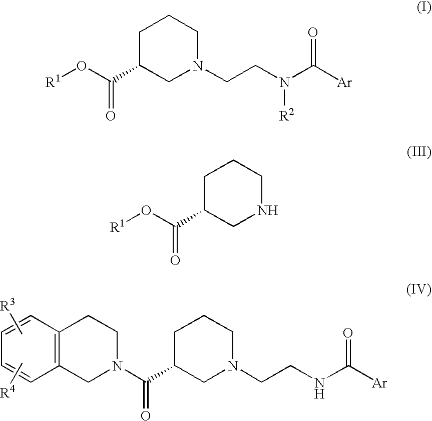

The invention further includes a compound where a part of the compound of the invention is labeled with radioisotope and a production process using a compound where a part of the compound of the invention is labeled with radioisotope.

The invention still further includes a salt, a hydrate and a solvate of the invention compound and a producing process using a compound in which salt, hydrate or solvate is formed. Specific examples of such a salt are a salt with inorganic acid such as hydrochloric acid, hydrobromic acid, hydroiodic acid, sulfuric acid, nitric acid or phosphoric acid, salt with organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid, p-toluenesulfonic acid, aspartic acid or glutamic acid, salt with inorganic base containing metal such as sodium, potassium, calcium or magnesium, salt with organic base such as methylamine, ethylamine, ethanolamine, lysine or ornithine and quaternary ammonium salt.

Process for Production

Process for production in accordance with the invention will be illustrated as hereunder.

Incidentally, in the production process according to the invention, there are some cases where, depending upon the type of functional group of the compound, it is effective in view of production technique to substitute the functional group, during the stage of a material or an intermediate, with an appropriate protective group or, in other words, a group which is easily able to be converted to the functional group. After that, the protective group is removed if necessary to give a desired compound. Examples of such a functional group include a carboxyl group and an amino group. Examples of such a protective group include those which are mentioned in the above-mentioned “Protective Groups in Organic Synthesis” (Third Edition) and they may be appropriately used depending upon the reaction condition.
(Production Process 1-1) Production Process for the Compound where R² is —H in the Compound Represented by the Formula (I)

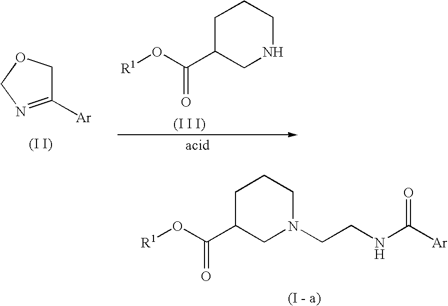

(In the formula, R¹ and Ar have the same meanings as defined already. They are the same hereinafter as well.)

This production process is a process for the production of a compound (I-a) which is the compound (I) of the invention where R² is —H by the reaction of a dihydrooxazole derivative (II) with a nipecotic acid derivative (III) under an acidic condition.

Examples of the acid which is able to be used in the present production process are mineral acid such as sulfuric acid, hydrochloric acid and hydrobromic acid, organic acid such as methanesulfonic acid, ethanesulfonic acid, benzenesulfonic acid, p-toluenesulfonic acid or a hydrate thereof, formic acid and acetic acid and Lewis acid such as boron trifluoride etherate and zinc chloride. In view of an industrial production, however, it is preferred to use an organic acid of sulfonic acid type or, particularly, p-toluenesulfonic acid or a hydrate thereof which is less expensive, easy to handle and able to proceed the reaction in a high yield. The use of p-toluenesulfonic acid hydrate is particularly preferred among the above. The acid may be used in 10 to 120 molar % to the compound (II) and, since there is a risk of making into an epi-compound when the reaction is conducted at a catalytic amount, it is preferred to conduct the reaction by addition of 1 equivalent to a somewhat excessive amount to the compound (II). To be specific, it is preferred to use 100 to 110 molar %.

The reaction is carried out in a solvent which is inert to the reaction, for example, an aromatic hydrocarbon such as benzene, toluene and xylene; a halogenated hydrocarbon such as dichloroemethane, dichloroethane and chloroform; an aprotic polar solvent such as dimethylformamide (DMF), dimethylacetamide (DMA) and dimethyl sulfoxide (DMSO); pyridine; water; a mixture solvent thereof; etc. under cooling to refluxing. A process conducting in a toluene solvent under refluxing is preferred. It is also possible to conduct in the absence of solvent.
(Production Process 1-2) Production Process for a Compound where R² in the Compound Represented by the Formula (I) is a Group other than —H 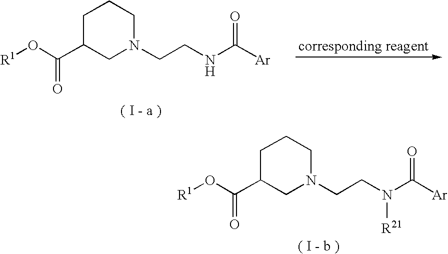
(In the formula, R²¹ is a group R² where —H is excluded. Hereinafter, it has the same meaning as well.)

The production process is a process for the production of a compound (I-b) which is the compound (I) of the invention where R² is other than —H by the action of the compound of the invention (I-a) produced by the production process 1-1 with the corresponding reagent.

With regard to the corresponding reagent, the reagent mentioned in the above “Protective Groups in Organic Synthesis” (Third Edition) or a reagent similar thereto may be used. With regard to the reaction, it is also possible to use the reaction mentioned in the above “Protective Groups in Organic Synthesis” (Third Edition) or a reaction similar thereto.
(Production Process 1-3) Process for the Production of the Compound of the Invention which is the Compound Represented by the Formula (I) where R¹ is —H 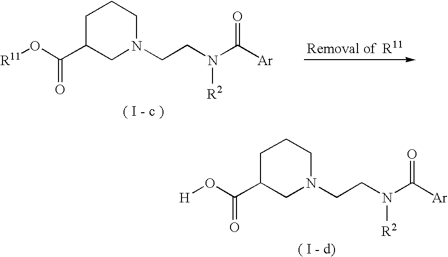
(In the formula, R¹¹ is the above-mentioned R¹ except the case-where it is —H and R² has the same meaning as above. Hereinafter, they have the same meanings as well.)

The production process is a process for the production of the compound (I-d) which is the compound of the invention (I) in which R¹ is —H by removal of R¹¹ of the compound (I-c) which is the compound of the invention (I) in which R¹ is other than —H produced by the production process 1-1 or the production process 1-2.

The reaction may be carried out for the compound (I-c) in a solvent which is inert to the reaction, for example, an aromatic hydrocarbon; ether such as diethyl ether, tetrahydrofuran (THF) and dioxane; a halogenated hydrocarbon; alcohol such as methanol (MeOH), ethanol (EtOH) and 2-propanol (i-PrOH); an aprotic polar solvent; pyridine; water; or a mixed solvent thereof; etc. in the presence of a mineral acid such as sulfuric acid, hydrochloric acid and hydrobromic acid, an organic acid such as formic acid and acetic acid or a base such as sodium hydroxide potassium hydroxide, potassium carbonate, sodium carbonate, cesium carbonate or ammonia under cooling to refluxing and the reaction temperature may be appropriately selected depending upon the reaction condition.

Besides the above-mentioned hydrolysis with an alkali and hydrolysis with an acid, it is also possible to utilize a catalytic reduction reaction where hydrogen is made to act in the presence of, for example, palladium-carried activated carbon or platinum catalyst particularly when R¹¹ is benzyl.

Besides the above, it is further possible to adopt a method mentioned in the above “Protective Groups in Organic Synthesis” (Third Edition) or a method similar thereto. A preferred example is that a compound where R¹¹ is lower alkyl is used as a starting material and is made to act with sodium hydroxide or potassium hydroxide in an alcohol corresponding to R¹¹ or a mixed solvent of the alcohol with water under cooling, under cooling to room temperature or under room temperature to heating.

It is still further possible that the compound (I-d) which is manufactured by the production process in which R² is —H is used as a starting material and is subjected to method of the production process 1-2 to give a compound of the invention compound (I) in which R¹ is —H and R² is other than —H.
(Production Process 2-1) Process for the Production of an Isoquinoline Derivative Represented by the Formula (IV) using a Compound which is the Compound (I) where R¹ is —H 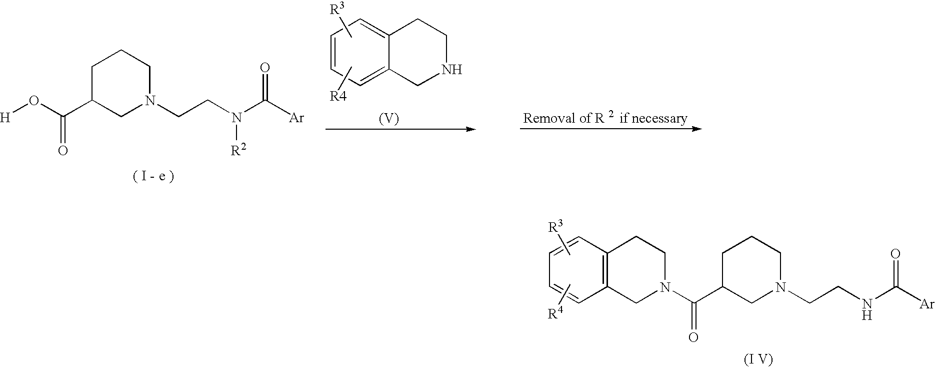
(In the formulae, R³ and R⁴ have the same meanings as above. Hereinafter, they are the same as well.)

This production process is a process for the production of an isoquinoline derivative represented by the formula (IV) in such a manner that the invention compound (I) wherein R¹ is —H produced by the production processes 1-1 to 1-3 are condensed with a tetrahydroisoquinoline derivative represented by the formula (V) and, when R² is a group other than —H, R² is removed therefrom.

In the present production process, it is also possible to use a reactive derivative of the compound (I-e) and examples of the reactive derivative are an acid halide such as acid chloride and acid bromide; an acid azide; an active ester with N-hydroxybenzotriazole, p-nitrophenol, N-hydroxysuccinimide, etc.; an acid anhydride of symmetric type; an alkyl halocarboxylate such as alkyl carbonic acid halide; a mixed acid anhydride with pivaloyl halide, p-toluenesulfonic acid halide, etc.; a mixed acid anhydride of phosphate type prepared by the reaction with diphenylphosphoryl chloride, diphenylphosphoryl azide, etc.; and the like.

When the compound (I-e) is made to react in a free acid or when the reaction is conducted without isolation of active ester, it is possible to use a condensing agent such as dicyclohexylcarbodiimide, 1,1′-carbonylbis-1H-imidazole, diethylphosphoryl cyanide, 1-ethyl-3-(3-dimethyiaminopropyl)carbodiimide hydrochloride (WSC.HCl), etc. Particularly in the present production process, a method in which reaction is conducted in the co-presence of active esterifying agent and condensing agent and an acid chloride method are applicable simply and easily and they are preferred.

Although being different depending upon the reactive derivative and the condensing agent used, the reaction may be carried out in a solvent which is inert to the reaction such as water, halogenated hydrocarbon, aromatic hydrocarbon, ether, alcohol, ester such as ethyl acetate (EtOAc), acetonitrile, aprotic polar solvent or a mixed solvent thereof under cooling, under cooling to room temperature or under room temperature to heating. It is sometimes advantageous in smoothly proceeding the reaction when the reaction is carried out in the presence of an organic base such as N-methylmorpholine, trimethylamine, triethylamine, diisopropylethylamine, N,N-dimethylaniline, pyridine, 4-(N,N-dimethylamino)pyridine, picoline and lutidine or an inorganic base such as potassium carbonate, cesium carbonate, sodium carbonate, sodium hydroxide and potassium hydroxide.

The compound (V) may also be used as its salt or, preferably, as a hydrochloride and, in that case, it is possible to use after desalting by the action of a base in the reaction system if necessary.
(Production process 2-2) Process for Production of an Isoquinoline Derivative Represented by the Formula (IV) using a Compound which is the Compound Represented by the Formula (I) where R¹ is a Group other than —H

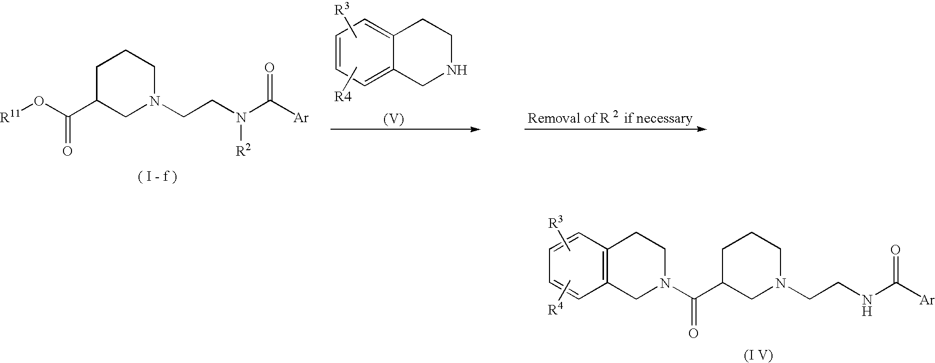

This production process is a process for production of an isoquinoline derivative represented by the formula (IV) in such a manner that a compound (I-f) which is the compound of the invention (I) manufactured by production processes 1-1 to 1-3 where R¹ is other than —H is condensed with a tetrahydroisoquinoline derivative represented by the formula (V) and, when R² is a group other than —H, R² is removed therefrom.

The reaction may be carried out in such a manner that the compound (I-f) and the compound (V) are dissolved or suspended in a solvent which is inert to the reaction such as aromatic hydrocarbon, ether, halogenated hydrocarbon, aprotic polar solvent, pyridine, water or a mixed solvent thereof under cooling to refluxing and the reaction temperature may be appropriately selected depending upon the reaction condition.

Specific examples are a method where a mixture containing the compound (I-f) and the compound (V) is heated and the resulting alcohol (R¹¹—OH) is evaporated, a method where an alkaline metal alkoxide such as sodium methoxide is made to act, a method where an alkaline metal strong base such as n-butyl lithium and sodium hydride is made to act, a method where a magnesium reagent such as Grignard reagent is made to act and a method where an aluminum reagent such as lithium aluminium hydride, diisobutyl aluminum hydride and trimethyl aluminum is made to act. It is also possible to refer to “Shin Jikken Kagaku Koza 14, Yuki Kagobutsu no Gosei to Hanno II (Experimental Chemistry 14, Synthesis and Reaction of Organic Compounds II) published by Maruzen (published on Dec. 20, 1977).

Incidentally, the same as in the case of the production process 2-1, it is also possible to use the compound (V) in its form of salt or, preferably, a hydrochloride and, at that time, it may be used after desalting using a base in the system if necessary.

### BEST MODE FOR CARRYING OUT THE INVENTION

Although the invention will be specifically illustrated hereunder by way of Examples, the invention is not limited to those Examples at all. Material compounds used in Examples are shown in Referential Examples. Purity of the compound was measured using a high-performance liquid chromatography (hereinafter, referred-to as HPLC).

Incidentally, in NMR, (CH₃)₄Si was used as an internal standard and, unless otherwise mentioned, it shows a peak value (ppm) in ¹H-NMR where DMSO-d₆ was used as a solvent for the measurement.

### REFERENTIAL EXAMPLE 1

Triethylamine (1.65 g) was dropped into a suspension (50 ml) of 3.75 g of 6,7-dimethoxy-1,2,3,4-tetrahydroisoquinoline monohydrochloride in THF under cooling with ice. The resulted mixture was stirred for 10 minutes under cooling with ice and then 3.74 g of

(R)-1-(tert-butoxycarbonyl)piperidine-3-carboxylic acid, 1.10 g of 1H-1,2,3-benzotriazol-1-ol (HOBt) and 3.44 g of WSC.HCl were added thereto successively. The reaction mixture was warmed to room temperature and stirred for one night and the solvent was evaporated in vacuo. The resulting residue was dissolved in chloroform and washed with a 1M aqueous solution of NaOH. The chloroform layer was dried over magnesium sulfate and filtered, then the solvent was evaporated in vacuo. The resulting residue was purified by a silica gel column chromatography (eluent (this is the same as hereinafter as well): chloroform) to give 6.60 g of tert-butyl (R)-3-(6,7-dimethoxy-1,2,3,4-tetrahydroisoquinoline-2-carbonyl)piperidine-1-carboxylate as a colorless amorphous substance.

FAB-MS m/z: 405 (M⁺+1).

### REFERENTIAL EXAMPLE 2

A 4M HC-EtOAc solution (12 ml) was dropped into an EtOH solution (30 ml) of 6.50 g of the compound of Referential Example 1 under cooling with ice. The reaction mixture was warmed to room temperature and stirred for one night and the resulting precipitate was filtered and dried to give 4.17 g of 6,7-dimethoxy-2-[(R)-piperidine-3-carbonyl]-1,2,3,4-tetrahydroisoquinoline monohydrochloride as white crystals. This was dissolved in chloroform and washed with a 1M aqueous solution of NaOH and the aqueous layer was extracted with chloroform twice. The organic layers were combined, dried over magnesium sulfate and filtered, then the solvent was evaporated in vacuo to give 3.10 of 6,7-dimethoxy-2-[(R)-piperidine-3-carbonyl]-1,2,3,4-tetrahydroisoquinoline as a colorless oily substance.

FAB-MS m/z: 305 (M⁺+1).

### REFERENTIAL EXAMPLE 3

2-(2-Bromoethyl)-1H-isoindole-1,3(2H)-dione (1.25 g) and 690 mg of potassium carbonate were added to a solution (25 ml) of 1.00 g of the compound of Referential Example 2 in acetonitrile at room temperature and, after that, the reaction mixture was stirred for one night at 70° C. The solvent was evaporated in vacuo and the resulting residue was dissolved in chloroform and washed with a saturated saline solution. The chloroform layer was dried over magnesium sulfate and filtered, then the solvent was evaporated in vacuo. The resulting residue was purified by silica gel column chromatography (chloroform: MeOH=93:7; after that, EtOAc) to give 1.45 g of 2{2-[(R)-3-(6,7-dimethoxy-1,2,3,4-tetrahydroisoquinoline-2-carbonyl)piperidino]ethyl}-1H-isoindole-1,3(2H)-dione as a yellow amorphous substance.

FAB-MS m/z: 478 (M⁺+1).

### REFERENTIAL EXAMPLE 4

A MeOH solution containing 40% of methylamine was added to 10 mL of MeOH solution of 1.35 g of the compound of Referential Example 3 at room temperature. The reaction mixture was stirred at room temperature for one night and then the solvent was evaporated in vacuo. The resulting residue was dissolved in chloroform and washed with an aqueous solution of NaHCO₃. The chloroform layer was dried over magnesium sulfate and filtered, then the solvent was evaporated in vacuo. The resulting residue was purified by a silica gel column chromatography (chloroform: MeOH: 28% aqueous ammonia=50:1:0 to 10:1:0.1) to give 830 mg of 2-[(R)-3-(6,7-dimethoxy-1,2,3,4-tetrahydroisoquinoline-2-carbonyl)piperidino]ethylamine as a yellow oily substance.

FAB-MS m/z: 348 (M⁺+1).

### REFERENTIAL EXAMPLE 5

A solution (5 ml) of 300 mg of 4-fluorobenzoyl chloride in acetonitrile was dropped into 10 ml of a solution of 650 mg of the compound of Referential Example 4 in acetonitrile under cooling with ice. The reaction mixture was warmed to room temperature and stirred for 4 hours. An aqueous solution of NaHCO₃ was added to the reaction mixture and, after stirring for 20 minutes, the solvent was evaporated in vacuo. To the resulting residue were added chloroform and an aqueous solution of NaHCO₃ followed by extracting with chloroform. The chloroform layer was dried over magnesium sulfate and filtered, then the solvent was evaporated in vacuo. The resulting residue was purified by a silica gel column chromatography (chloroform: MeOH=50:1 to 10:1) and purified by an active alumina column chromatography (hexane: EtOAc=1:1, then EtOAc and EtOAc: MeOH=50:1) to give 600 mg of a colorless oily substance. The oily substance was dissolved in 10 ml of EtOH and 180 mg of 85% phosphoric acid was added thereto. The reaction mixture was heated to completely dissolve and cooled to room temperature. The resulting crystals were filtered and recrystallized from 95% EtOH-water to give 632 mg of (−)-N-{2-[(R)-3-(6,7-dimethoxy-1,2,3,4-tetrahydroisoquinoline-2-carbonyl)piperidino]ethyl}-4-fluorobenzamide monophosphate as colorless crystals.

NMR: δ 1.25-1.50 (1H, m), 1.53-1.76 (3H, m), 2.15-2.80 (6H, m), 2.95-3.10 (3H m), 3.40-3.50 (2H, m), 3.60-3.75 (8H, m), 4.50 (1H, q), 4.63 (1H, q), 6.73 (1H, s), 6.78, 6.85 (1H, s in combination), 7.29 (2H, t), 7.91-7.95 (2H, m), 8.60 (1H, br).

FAB-MS m/z: 470 (M⁺+1).

### REFERENTIAL EXAMPLE 6

Ethyl (R)-piperidine-3-carboxylate L-tartrate (79.0 g) was dissolved in 150 ml of water and 100 ml of chloroform. To the reaction solution was added 75 ml of an aqueous 8 M KOH under cooling with ice followed by extracting with chloroform. The chloroform layer was dried over magnesium sulfate and filtered, then the solvent was evaporated in vacuo. The resulting residue was added to 400 ml of solution of 69.0 g of tert-butyl (2-bromoethyl)carbamate in acetonitrile at room temperature together with 42.6 g of potassium carbonate. The reaction mixture was stirred at 60° C. for one night and an insoluble matter was filtered off. The filtrate was evaporated in vacuo and the resulting residue was dissolved in 500 ml of EtOAc. This was extracted for three times with a saturated aqueous solution of citric acid and the aqueous layer was cooled with ice. Its pH was adjusted to about 10 with a 8 M aqueous solution of KOH followed by extracting with chloroform for four times. The chloroform layer was dried over magnesium sulfate and filtered, then the solvent was evaporated in vacuo to give 72.7 g of ethyl

(R)-1-{2-[(tert-butoxycarbonyl)amino]ethyl}piperidine-3-carboxylate as a light yellow oily substance.

NMR (CDCl₃): δ 1.24 (3H, t), 1.45 (9H, s), 1.47-1.58 (2H, m), 1.67-1.77 (2H, M), 1.80-1.94 (1H, m), 2.00-2.15 (1H, m), 2.26-2.38 (1H, m), 2.50-2.60 (1H, m), 2.60-2.70 (1H, m), 2.75-2.90 (1H, m), 3.15-3.27 (2H, m), 4.16 (2H, q).

FAB-MS m/z: 301 (M⁺+1).

### REFERENTIAL EXAMPLE 7

4 M solution of HCl in EtOAc (150 ml) was dropped into 150 ml of ethanolic solution of 72.1 g of the compound of Referential Example 6 under cooling with ice-bath. The reaction mixture was warmed to room temperature and stirred for one night and the solvent was evaporated in vacuo. The resulting residue was dissolved in water, pH was adjusted to about 10 with a 8 M aqueous solution of KOH under cooling with ice and the aqueous layer was extracted with chloroform for four times. The organic layers were combined, dried over magnesium sulfate and filtered, then the solvent was evaporated in vacuo. Into a solution (200 ml) of the resulting light yellow oily substance in THF was dropped 40.0 g of 4-fluorobenzoyl chloride at 10° C. or a lower temperature under cooling with ice-bath. The reaction mixture was stirred for 2 hours under cooling with ice. EtOAc was added to the reaction mixture followed by extracting with a 1M aqueous solution of HCl for two times. The aqueous layer was adjusted to pH about 9 with a 8 M aqueous solution of KOH under cooling with ice. The aqueous layer was extracted with chloroform for three times, the chloroform layer was dried over magnesium sulfate and filtered, then the solvent was evaporated in vacuo. The resulting residue was crystallized from diisopropyl ether to give 26.33 g of ethyl

(R)-1-{2-[(4-fluorobenzoyl)amino]ethyl}piperidine-3-carboxylate as colorless crystals.

NMR (CDCl₃): δ 1.23 (3H, t), 1.50-1.62 (1H, m), 1.66-1.86 (3H, m), 2.25-2.37 (1H, m), 2.50-2.75 (6H, m), 3.44-3.52 (1H, m), 3.57-3.66 (1H, m), 4.04-4.20 (2H, m), 7.00 (1H, br), 7.06-7.13 (2H, m), 7.81-7.88 (2H, m).

FAB-MS m/z: 323 (M⁺+1).

### REFERENTIAL EXAMPLE 8

A 1M aqueous solution (177 ml) of NaOH was dropped into 100 ml of an ethanolic solution of 37.94 g of the compound prepared in the method of Referential Example 7 at room temperature. After stirring at room temperature for 1 hour, it was cooled with ice. An aqueous solution of hydrochloric acid was added to the reaction mixture to adjust the pH to acidic and the solvent was azeotropically evaporated with toluene in vacuo. DMF (250 ml) was added to the resulting residue and, at 10° C., 21.66 g of 6,7-dimethoxy-1,2,3,4-tetrahydroisoquinoline (prepared by desalting of 6,7-dimethoxy-1,2,3,4-tetrahydroisoquinoline monohydrochloride), 7.97 g of HOBt and 27.14 g of WSC.HCl successively. The reaction mixture was stirred for 3 hours at room temperature and poured into a mixed solution of EtOAc and water followed by extracting with a 1M aqueous solution of HCl for two times. The combined aqueous layer was adjusted to pH about 10 using an aqueous solution of NaOH under cooling with ice. The aqueous layer was extracted with chloroform for three times, the combined chloroform layer was dried over magnesium sulfate and filtered, then the solvent was evaporated in vacuo. The resulting residue was dissolved in 500 ml of EtOH and 13.65 g of 85% phosphoric acid was added thereto. The compound of Referential Example 5 was added as seed crystals and the mixture was stirred at room temperature for 3 days. The resulting crystals were filtered to give 44.25 g of (−)-N-{2-[(R)-3-(6,7-dimethoxy-1,2,3,4-tetrahydroisoquinoline-2-carbonyl)-piperidino]ethyl}-4-fluorobenzamide monophosphate as colorless crystals.

### REFERENTIAL EXAMPLE 9

The compound (100 g) manufactured by the method of Referential Example 8 was recrystallized from 2,200 ml of 95% EtOH-water to give 89.32 g of (−)-N-{2-[(R)-3-(6,7-dimethoxy-1,2,3,4-tetrahydroisoquinoline-2-carbonyl)piperidino]ethyl}-4-fluorobenzamide monophosphate as colorless crystals.

NMR: δ 1.33-1.50 (1H, m), 1.65-1.86 (3H, m), 2.35-2.50 (1H, m), 2.50-2.60 (1H, m), 2.62-2.69 (1H, m), 2.72-2.90 (3H, m), 3.09-3.23 (3H, m), 3.48-3.58 (2H, m), 3.62-3.75 (8H, m), 4.50 (1H, q), 4.63 (1H, q), 6.72 (1H, s), 6.78, 6.88 (1s in combination), 7.24-7.32 (2H, m), 7.94-8.00 (2H, m), 8.80 (1H, br).

FAB-MS m/z: 470 (M⁺+1).

### REFERENTIAL EXAMPLE 10

To a solution including 900 ml of water and 414.9 g of potassium carbonate was added 307.5 g of 2-bromoethylamine monohydrobromide with stirring at −5° C. or a lower temperature. EtOAc (750 ml) was added to the reaction mixture and then 238.0 g of 4-fluorobenzoyl chloride was added thereto with stirring at 12° C. or a lower temperature. To this was added 150 ml of EtOAc. The reaction was monitored by HPLC measurement whereupon the production of N-(2-bromoethyl)-4-fluorobenzamide having a purity of 93.0% was confirmed (the confirmation was conducted by means of comparison of retention time in HPLC with commercially available N-(2-bromoethyl)-4-fluorobenzamide).

### REFERENTIAL EXAMPLE 11

The reaction mixture of Referential Example 10 was heated and stirred at about 50° C. for 4 hours. To the reaction mixture was added 450 ml of toluene, the mixture was allowed to stand and separation was conducted at about 35° C. An organic layer was washed with 900 ml of water. The organic layer was concentrated in vacuo and then dried in vacuo to give 240.14 g of 2-(4-fluorophenyl)-4,5-dihydrooxazole having a purity of 99%.

NMR: δ 3.96 (2H, t), 4.41 (2H, t), 7.28-7.34 (2H, m), 7.89-7.95 (2H, m).

FAB-MS m/z: 166 (M⁺+1).

### EXAMPLE 1

To 120.0 g of the compound of Referential Example 11 were added 1,200 ml of toluene and 137.07 g of ethyl (R)-piperidine-3-carboxylate. Ethyl (R)-piperidine-3-carboxylate was washed with 600 ml of toluene. After that, 145.1 g of p-toluenesulfonic acid monohydrate was added thereto followed by washing with 600 ml of toluene. The reaction mixture was heated and the solvent was distilled at ordinary pressure to evaporate 600 ml thereof. After that, the reaction mixture was stirred for 27 hours under reflux. After the mixture solution was cooled, 960 ml of EtOAc and 960 ml of 4% (w/v) aqueous solution of NaHCO₃ were added. The reaction mixture was allowed to stand and separation was conducted at about 35° C. The resulted organic layer was washed twice with 960 ml of a 4% (w/v) aqueous solution of NaHCO₃. The organic layer was concentrated in vacuo to give ethyl

(R)-1-{2-[(4-fluorobenzoyl)amino]ethyl}piperidine-3-carboxylate having a purity of 82.8%.

NMR: δ 1.16 (3H, t), 1.35-1.80 (4H, m), 2.05-2.10 (1H, m), 2.20-2.26 (1H, m), 2.45-2.51 (3H, m), 2.65-2.70 (1H, m), 2.85-2.90 (1H, m), 3.30-3.38 (2H, m), 4.05 (2H, q), 7.25-7.33 (2H, m), 7.85-7.95 (2H, m), 8.35-8.40 (1H, m).

FAB-MS m/z: 323 (M⁺+1).

### EXAMPLE 2

To 230 g of the compound of Example 1 was added 690 ml of EtOH and then 345 ml of water was added thereto. After cooling, an aqueous solution of NaOH (42.8 g NaOH/480 ml water) was added followed by stirring for 2 hours at 25° C. or a lower temperature. After cooling, concentrated hydrochloric acid was added to the reaction mixture to adjust to pH 3.0. This solution was concentrated in vacuo, 1,000 ml of toluene was added to the residue and the mixture was concentrated in vacuo to give

(R)-1-{2-[(4-fluorobenzoyl)amino]ethyl}piperidine-3-carboxylic acid having a purity of 86.3%.

NMR (90° C.): δ 1.46-1.60 (1H, m), 1.79-2.05 (3H, m), 2.75-3.60 (7H, m), 3.68 (2H, q), 7.20-7.27 (2H, m), 7.95-8.03 (2H, m), 8.74 (1H, brs).

FAB-MS m/z: 295 (M⁺+1).

### EXAMPLE 3

To 206.4 g of the compound of Example 2 were added 810 ml of DMF and 120.8 g of 6,7-dimethoxy-1,2,3,4-tetrahydroisoquinoline monohydrochloride followed by stirring and cooling. After that 53.22 g of triethylamine was added at 12° C. or a lower temperature and then 217 ml of DMF was added. Then 21.32 g of HOBT was added at 5° C. or a lower temperature and then 121.0 g of WSC.HCl was added at 5° C. or a lower temperature. The reaction mixture was stirred at 0 to 4° C. for 15.5 hours. To the reaction mixture were added 340 ml of water, 2,000 ml of EtOAc and 550 ml of a 8% (w/v) aqueous solution of NaOH followed by separating. EtOAc (1,000 ml) was added to the aqueous layer followed by separating. After that, the organic layers were mixed and washed for two times with 700 ml of a 8% (w/v) aqueous solution of NaOH and 300 ml of water. After the organic layer was washed with 900 ml of water, it was concentrated in vacuo to give (−)-N-{2-[

(R)-3-(6,7-dimethoxy-1,2,3,4-tetrahydroisoquinoline-2-carbonyl)piperidino]ethyl}-4-fluorobenzamide having a purity of 83.9%.

NMR (CDCl₃): δ 1.54-1.68 (2H, m), 1.75-1.87 (2H, m), 2.05-2.95 (9H, m), 3.45-3.63 (2H, m), 3.70 (1H, t), 3.75-3.82 (1H, m), 3.84 (3H, s), 3.85 (3H, s), 4.59 (1H, br), 4.62 (1H, br), 6.55-6.65 (2H, m), 6.95 (1H, br), 7.11 (2H, t), 7.77-7.88 (2H, m).

FAB-MS m/z: 470 (M⁺+1).

[α]_{D}²⁰: −4.16° (solvent: MeOH).

### EXAMPLE 4

To 243.94 g of the compound of Example 3 was added 4,580 ml of EtOH. Then 59.95 g of 85% phosphoric acid was added at about 30° C. After that, 57.5 ml of water was used for washing. To this solution was added the compound of Referential Example 9 as seed crystals followed by cooling. The crystals separated out therefrom were filtered, washed with EtOH and dried in vacuo to give 243.22 g of (−)-N-{2-[(R)-3-(6,7-dimethoxy-1,2,3,4-tetrahydroisoquinoline-2-carbonyl)piperidino]ethyl}-4-fluorobenzamide monophosphate (crude crystals) having a purity of 97%.

NMR: δ 1.30-1.50 (1H, m), 1.60-185 (3H, m), 2.30-2.90 (6H, m), 3.00-3.20 (3H, m), 3.40-3.57 (2H, m), 3.60-3.78 (8H, m), 4.50 (1H, q), 4.63 (1H, q), 6.73 (1H, s), 6.78, 6.86 (1H, s in combination), 7.20-7.35 (2H, m), 7.87-8.01 (2H, m), 8.65-8.77 (1H, m).

FAB-MS m/z: 470 (M⁺+1).

[α]_{D}²⁰: −20.1° (solvent: water)

### EXAMPLE 5

To 220.0 g of the compound of Example 4 were added 2,200 ml of EtOH and 250 ml of water. The mixture was heated at nearly a refluxing temperature and, after crude crystals were dissolved, the resulted solution was filtered. The filtrate was heated with stirring and cooled. The compound of Referential Example 9 was added as seed crystals followed by cooling. The crystals separated out therefrom were filtered, washed with EtOH and dried in vacuo to give 179.75 g of (−)-N-{2-[(R)-3-(6,7-dimethoxy-1,2,3,4-tetrahydroisoquinoline-2-carbonyl)-piperidino]ethyl}-4-fluorobenzamide monophosphate having a purity of 99.5%.

NMR: δ 1.30-1.50 (1H, m), 1.52-1.85.(3H, m), 2.40-2.90 (6H, m), 2.95-3.30 (3H, m), 3.53 (2H, br), 3.60-3.75 (8H, m), 4.50 (1H, q), 4.64 (1 H, q), 6.72 (1H, s), 6.78, 6.88 (1H, s in combination), 7.28 (2H, t), 7.96 (2H, t), 8.81 (1H, br).

FAB-MS m/z: 470 (M⁺+1).

[α]_{D}²⁰: −20.7° (solvent: water)

Result of elementary analysis of this compound is shown in Table 1.

TABLE 1
Result of Elementary Analysis of Compound of Example 5

| | C (%) | H (%) | N (%) | O (%) | F (%) | P (%) |
|---|---|---|---|---|---|---|
| Calculated | 55.02 | 6.22 | 7.40 | 22.55 | 3.35 | 5.46 |
| Found | 55.02 | 6.11 | 7.33 | | 3.08 | 5.44 |

To a solution comprising 110 liters of water and 36.8 kg of potassium carbonate was added 27.3 kg of 2-bromoethylamine monohydrobromide at −5° C. or a lower temperature with stirring. To the reaction mixture was added 100 liters of EtOAc and then 21.1 kg of 4-fluorobenzoyl chloride was added thereto at 5° C. or a lower temperature with stirring. To this was added 10 liters of EtOAc. The reaction mixture was monitored by HPLC measurement to confirm the production of N-(2-bromoethyl)-4-fluorobenzamide having a purity of 85.2%.

### REFERENTIAL EXAMPLE 13

The reaction mixture of Referential Example 12 was heated and stirred at 45 to 52° C. for 4 hours. To the reaction mixture was added 40 liters of toluene and the mixture was allowed to stand to separate at about 35° C. The organic layer was washed with 80 liters of water. As a result, a solution of 2-(4-fluorophenyl)-4,5-dihydrooxazole having a purity of 98% in toluene-EtOAc was prepared.

### EXAMPLE 6

To the toluene-EtOAc solution prepared in Referential Example 13 were added 440 liters of toluene, 25.1 kg of ethyl (R)-piperidine-3-carboxylate and 26.6 kg of p-toluenesulfonic acid monohydrate, the mixture was heated and the solvent was distilled at atmospheric pressure to evaporate 260 liters. After that, stirring was conducted with refluxing for 32 hours. After the reaction mixture was cooled, 260 liters of EtOAc and 180 liters of a 4% (w/v) aqueous solution of NaHCO₃ were added. The reaction mixture was allowed to stand and separation was conducted at about 30° C. The organic layer was washed with 180 liters of a 4% (w/v) aqueous solution of NaHCO₃ twice. The organic layer was concentrated in vacuo to give ethyl (R)-1-{2-[(4-fluorobenzoyl)amino]ethyl}piperidine-3-carboxylate having a purity of 86.7%.

### EXAMPLE 7

To the compound prepared in Example 6 was added 260 liters of EtOH and then 64 liters of water was added thereto. After cooling, an aqueous solution of NaOH (8.0 kg NaOH/90 liters water) was added thereto followed by stirring at 25° C. or a lower temperature for 2 hours. After cooling, concentrated hydrochloric acid was added to the reaction to adjust pH to 3.06. This solution was concentrated in vacuo, 190 liters of toluene was added to the residue and the mixture was concentrated in vacuo. To the residue was added 190 liters of toluene followed by concentrating in VaCuo. To the residue was added 190 liters of toluene followed by concentrating in vacuo to give

(R)-1-{2-[(4-fluorobenzoyl)amino]ethyl}piperidine-3-carboxylic acid having a purity of 90.4%.

### EXAMPLE 8

To the compound prepared in Example 7 were added 200 liters of DMF and 23.0 kg of 6,7-dimethoxy-1,2,3,4-tetrahydroisoquinoline monohydrochloride followed by stirring and cooling. Then 10.1 kg of triethylamine was added at 10° C. or a lower temperature and 4.0 kg of HOBt was added thereto at 5° C. or a lower temperature. After that, 23.0 kg of WSC.HCl was added at 5° C. or a lower temperature followed by stirring at −4 to 4° C. for one night. To the reaction mixture were-added 64 liters of water, 380 liters of EtOAc and 105 liters of a 8% (w/v) aqueous solution of NaOH followed by separating. To the aqueous layer was added 190 liters of EtOAc followed by separating. After that, organic layers were combined and washed for two times with 130 liters of a 8% (w/v) aqueous solution of NaOH and 57 liters of water. After the organic layer was washed with 170 liters of water, it was concentrated in vacuo to give (−)-N-{2-[(R)-3-(6,7-dimethoxy-1,2,3,4-tetrahydroisoquinoline-2-carbonyl)piperidino]ethyl}-4-fluorobenzamide having a purity of 79%. EtOH (140 liters) was added thereto to dissolve.

### EXAMPLE 9

To the ethanolic solution prepared in Example 8 was added 330 liters of EtOH. After that, 11.5 kg of 85% phosphoric acid and 52 liters of water were added thereto followed by heating and the resulting solution was filtered. The filtrate was heated with stirring and cooled and the compound of Example 5 was added as seed crystals followed by cooling. The crystals separated out therefrom were filtered, washed with EtOH and dried in vacuo to give 36.51 kg of (−)-N-{2-[(R)-3-(6,7-dimethoxy-1,2,3,4-tetrahydroisoquinoline-2-carbonyl)piperidino]ethyl}-4-fluorobenzamide monophosphate having a purity of 98.9%.

NMR: δ 1.30-1.50 (1H, m), 1.60-1.85 (3H, m), 2.30-2.85 (6H, m), 3.05-3.20 (3H, m), 3.40-3.57 (2H, m), 3.60-3.77 (8H, m), 4.50 (1H, q), 4.63 (1H, q), 6.72 (1H, s), 6.77, 6.86 (1H, s in combination), 7.27 (2H, t), 7.88-8.00 (2H, m), 8.70 (1H, br).

FAB-MS m/z: 470 (M⁺+1).

[α]_{D}²⁰: −20.8° (solvent: water)

Result of elementary analysis of this compound was the same as that for the compound of Example 5.

### REFERENTIAL EXAMPLE 14

To a solution including 470 ml of water and 156.9 g of potassium carbonate was added 116.3 g of 2-bromoethylamine monohydrobromide with stirring at −5° C. or a lower temperature. To the reaction mixture was added 420 ml of EtOAc and then 90.0 g of 4-fluorobenzoyl chloride was added thereto with stirring at 5° C. or a lower temperature. To this was added 43 ml of EtOAc. The reaction mixture was monitored by HPLC measurement to confirm the production of N-(2-bromoethyl)-4-fluorobenzamide having a purity of 95.8%.

### REFERENTIAL EXAMPLE 15

The reaction mixture of Referential Example 14 was heated and stirred for 3 hours at 48 to 53° C. To the reaction mixture was added 175 ml of toluene and the mixture was allowed to stand to separate at 30 to 35° C. The organic layer was washed with 340 ml of water to give a solution of 2-(4-fluorophenyl)-4,5-dihydrooxazole having a purity of 99.4% in toluene-EtOAc.

### EXAMPLE 10

To the toluene-EtOAc solution prepared in Referential Example 15 were added 2,250 ml of toluene, 107.1 g of ethyl (R)-piperidine-3-carboxylate and 113.3 g of p-toluenesulfonic acid monohydrate followed by heating and the solvent was distilled at an ordinary pressure to evaporate 1,500 ml. After that, the mixture was stirred for 32 hours under reflux. After the reaction mixture was cooled, 1,130 ml of EtOAc and 1,600 ml of a 4% (w/v) aqueous solution of NaHCO₃ were added. The reaction mixture was allowed to stand and separation was conducted at about 35° C. The organic layer was washed twice with 750 ml of a 4% (w/v) aqueous solution of NaHCO₃. The organic layer was divided into three. To one of them was added 100 ml of EtOAc followed by concentrating in vacuo to give ethyl

(R)-1-{2-[(4-fluorobenzoyl)amino]ethyl}piperidine-3-carboxylate having a purity. of 89.6%.

### EXAMPLE 11

To the compound prepared in Example 10 was added 370 ml of EtOH and then 91 ml of water was added thereto. After cooling, an aqueous solution of NaOH (11.35 g NaOH/128 ml water) was added followed by stirring at 25° C. or a lower temperature for 2 hours. After cooling, concentrated hydrochloric acid was added to the reaction mixture to make pH 3.22. The solution was concentrated in vacuo and 270 ml of toluene was added to the residue followed by concentrating in vacuo. To the resulting residue was added 270 ml of toluene followed by concentrating in vacuo. To the resulting residue was added 270 ml of toluene followed by concentrating in vacuo to give (R)-1-{2-[(4-fluorobenzoyl)amino]ethyl}piperidihe-3-carboxylic acid having a purity of 91.3%.

### EXAMPLE 12

To the compound prepared in Example 11 were added 280 ml of DMF and 38.3 g of 6,7-dimethoxy-1,2,3,4-tetrahydroisoquinoline monohydrochloride followed by stirring and cooling. Then 16.87 g of triethylamine was added at 15° C. or a lower temperature and 6.76 g of HOBt was added at 5° C. or a lower temperature. After that, 38.4 g of WSC.HCl was added at 5° C. or a lower temperature followed by stirring at 0 to 5° C. for one night. To the reaction mixture were added 107 ml of water, 630 ml of EtOAc and 176 ml of a 8% (w/v) aqueous solution of NaOH to separate. To the aqueous layer was added 320 ml of EtOAc to separate. After that, the organic layers were mixed and washed for two times with 220 ml of a 8% (w/v) aqueous solution of NaOH and 96 ml of water. The organic layer was washed with 285 ml of water and concentrated in vacuo to give (−)-N-{2-[(R)-3-(6,7-dimethoxy-1,2,3,4-tetrahydroisoquinoline-2-carbonyl)piperidino]ethyl}-4-fluorobenzamide having a purity of 84.8%. To this was added 235 ml of EtOH to dissolve.

### EXAMPLE 13

EtOH (545 ml) was added to an ethanolic solution prepared in Example 12. After that, 19.22 g of 85% phosphoric acid and 86 ml of water were added followed by heating and the solution was filtered. The filtrate was heated with stirring and cooled and the compound of Example 5 was added as seed crystals followed by cooling. The crystals separated out therefrom were filtered, washed with ethanol and dried in vacuo to give 70.78 g of (−)-N-{2-[(R)-3-(6,7-dimethoxy-1,2,3,4-tetrahydroisoquinoline-2-carbonyl)piperidino]ethyl}-4-fluorobenzamide monophosphate having a purity of 99.2%.

NMR, MS and elementary analysis of this compound were the same as those of the compounds of Examples 5 and 9. Result of elementary analysis of this compound is shown in Table 2.

TABLE 2
Result of Elementary Analysis of Compound of Example 13

| | C (%) | H (%) | N (%) | O (%) | F (%) | P (%) |
|---|---|---|---|---|---|---|
| Calculated | 55.02 | 6.22 | 7.40 | 22.55 | 3.35 | 5.46 |
| Found | 54.80 | 6.17 | 7.39 | | 3.33 | 5.47 |

The overall yield from 4-fluorobenzoyl chloride of Referential Example 14 to the compound of Example 13 was 65.9%.

### EXAMPLE 14

To one of the organic layers divided into three in Example 10 was added 100 ml of EtOAc followed by concentrating in vacuo. To the residue after concentration were added 120 ml of diisopropyl ether and 120 ml of n-heptane. It was heated to dissolve, the compound of Referential Example 7 was added as seed crystals and crystallization was conducted at about −4° C. The filtered crystals were washed with a mixed solution of diisopropyl ether with n-heptane and dried in vacuo to give 53.31 g of ethyl (R)-1-{2-[(4-fluorobenzoyl)amino]ethyl}piperidine-3-carboxylate having a purity of 94.4%.

The overall yield from 4-fluorobenzoyl chloride of Referential Example 14 to the compound of Example 14for obtaining as crystals was 87.4%.

NMR: δ 1.16 (3H, t), 1.30-1.90 (4H, m), 2.05-2.18 (1H, m), 2.20-2.30 (1H, m), 2.42-2.58 (3H, m), 2.62-2.75 (1H, m), 2.82-2.95 (1H, m), 3.30-3.40 (2H, m), 4.05 (2H, q), 7.25-7.33 (2H, m), 7.85-7.95 (2H, m), 8.30-8.40 (1H, m).

ESI-MS (positive mode) m/z: 323 (M+H)⁺.

### EXAMPLE 15

To 51.2 g of the compound of Example 14 was added 310 ml of EtOH and then 76 ml of water was further added thereto. After cooling, an aqueous solution of NaOH (9.50 g NaOH/105 ml water) was added followed by stirring for 2 hours at 25° C. or a lower temperature. After cooling, concentrated hydrochloric acid was added to the reaction mixture to adjust to pH 3.22 followed by concentrating. To the residue was added 230 ml of toluene followed by concentrating in vacuo. Toluene (230 ml) was further added thereto followed by concentrating in vacuo and, once again, 230 ml of toluene was added followed by concentrating in vacuo to give

(R)-1-{2-[(4-fluorobenzoyl)amino]ethyl}piperidine-3-carboxylic acid having a purity of 93.8%.

### EXAMPLE 16

To the compound prepared in Example 1.5 were added 230 ml of DMF and 34.3 g of 6,7-dimethoxy-1,2,3,4-tetrahydroisoquinoline monohydrochloride followed by stirring and cooling. Further, 15.11 g of triethylamine was added at 15° C. or a lower temperature and then 6.04 g of HOBt was added at 5° C. or a lower temperature. Furthermore, 34.35 g of WSC.HCl was added at 5° C. or a lower temperature followed by stirring at 0 to 5° C. for one night. To the reaction mixture were added 96 ml of water, 570 ml of EtOAc and 158 ml of a 8% (w/v) aqueous solution of NaOH followed by separating. To the aqueous layer was added 285 ml of EtOAc to separate and the resulting organic layers were mixed and washed for two times with 196 ml of 8% (w/v) aqueous solution of NaOH and 86 ml of water. It was further washed with 255 ml of water and concentrated in vacuo to give (−)-N-{2-[(R)-3-(6,7-dimethoxy-1,2,3,4-tetrahydroisoquinoline-2-carbonyl)piperidino]ethyl}-4-fluorobenzamide having a purity of 91.8%. This was dissolved in 210 ml of EtOH to prepare an ethanolic solution.

### EXAMPLE 17

To the ethanolic solution prepared in Example 16 was further added 490 ml of EtOH, then 17.23 g of 85% phosphoric acid and 77 ml of water were added thereto and the mixture was heated and filtered. The filtrate was heated with stirring and cooled and the compound of Example 5 was added as seed, crystals. It was further cooled and crystals separated out therefrom were filtered, washed with EtOH and dried in vacuo to give 68.90 g of (−)-N-{2-[(R)-3-(6,7-dimethoxy-1,2,3,4-tetrahydroisoquinoline-2-carbonyl)piperidino]ethyl}-4-fluorobenzamide monophosphate having a purity of 99.6%.

NMR, MS and elementary analysis of this compound were the same as those of the compounds of Examples 5, 9 and 13. Result of elementary analysis of this compound is shown in Table 3.

TABLE 3
Result of Elementary Analysis of Compound of Example 17

| | C (%) | H (%) | N (%) | O (%) | F (%) | P (%) |
|---|---|---|---|---|---|---|
| Calculated | 55.02 | 6.22 | 7.40 | 22.55 | 3.35 | 5.46 |
| Found | 54.75 | 6.19 | 7.37 | | 3.31 | 5.45 |

The overall yield from 4-fluorobenzoyl chloride of Referential Example 14 to the compound of Example 17 was 66.8% and the overall yield from ethyl (R)-1-{2-[(4-fluorobenzoyl)amino]ethyl}piperidine-3-carboxylate of Example 14 to the compound of Example 17 was 76.4%.

### EXAMPLE 18

To 2.01 g of 6,7-dimethoxy-1,2,3,4-tetra hydroisoquinoline was added 15 ml of toluene, the mixture was cooled and 10 ml of diisobutylaluminum hydride (a solution of 1.01 mol/liter of toluene) was added under a nitrogen atmosphere. After stirring at about 20° C., 2.90 g of ethyl (R)-1-{2-[(4-fluorobenzoyl)amino]ethyl}piperidine-3-carboxylate was added followed by stirring at about 60° C. for one night. After cooling the reaction mixture, 6 ml of MeOH, 36 ml of a 1M aqueous solution of NaOH and 50 ml of EtOAc were added and the mixture was filtered using Celite and washed with 120 ml of EtOAc. The organic layer of the filtrate was washed with 60 ml of a 1 M aqueous solution of NaOH and then washed with 60 ml of water twice. The organic-layer was concentrated in vacuo, the resulting residue was dissolved in 30 ml of MeOH and, after cooling, 6 ml of a 1M aqueous solution of NaOH was added. The solution was stirred at 15° C. or a lower temperature and concentrated in vacuo, 80 ml of EtOAc and 80 ml of water were added to the residue and the resulting organic layer was washed with 80 ml of water. To the resulting organic layer was added 150 ml of a 1M aqueous solution of HCl. Chloroform (300 ml) was added to the aqueous layer and, after cooling, 210 ml of a 1M aqueous solution of NaOH was added. The resulting organic layer was washed with 300 ml of water and concentrated in vacuo to give 3.68 g of (−)-N-{2-[(R)-3-(6,7-dimethoxy-1,2,3,4-tetrahydroisoquinoline-2-carbonyl)piperidino]ethyl}-4-fluorobenzamide having a purity of 90%.

NMR and MS of this compound were the same as those of the compound of Example 3.

### INDUSTRIAL APPLICABILITY

In accordance with the invention, it is now possible to prepare, in high purity and also in high yield, an isoquinoline derivative which has an inhibitory action for I_{f} current and shows a strong and specific activity lowering a heart beat and decreasing oxygen consumption of heart muscle in a selective manner whereby it is useful as a preventive and/or treating agent for diseases of circulatory system such as ischemic, heart diseases (e.g., angina pectoris and myocardial infarction), congestive heart failure, arrhythmia, etc. Thus, the overall yield for isolating the isoquinoline derivative as its salt from a substituted benzoyl halide which is a known compound is higher than 65%. When a salt of (−)-N-{2-[(R)-3-(6,7-dimethoxy-1,2,3,4-tetrahydroisoquinoline-2-carbonyl)piperidino]ethyl}-4-fluorobenzamide is manufactured according to a method mentioned in Patent Document 1 which is a known method for the manufacture of isoquinoline derivatives, the overall yield is about 29% as shown in Referential Example 1 to Referential Example 5. Thus, when the production process of the invention is adopted for the manufacture of an isoquinoline derivative, the isoquinoline derivative is now able to be manufactured in a high yield whereby productivity and energy efficiency are improved and that is less expensive in terms of economy as well.

Further, in the production process of the invention, purification by column chromatography is not needed for the manufacture of an isoquinoline derivative and, therefore, it is quite efficient in view of an industrial production. Furthermore, deprotection reaction which results in a reduction in productivity in an industrial production is not needed and, in addition, the manufacture without the use of a solvent of halogen type is possible whereby it is excellent in terms of preservation of the environment and security.

Consequently, the production process according to the invention is a very good process for the production of an isoquinoline derivative and a benzamide derivative of the invention which is an intermediate for the above production process is a very useful intermediate for the production of the isoquinoline derivative. In addition, an N-alkylation reaction for dihydrooxazole using an acid which is a production process according to the invention is highly general-purpose and is a very important reaction.

1. A benzamide derivative represented by the formula (I) or a salt thereof. 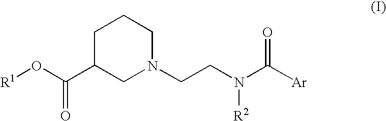
[In the formula, R¹ is —H or an ester residue; R² is —H or a protective group of amino group; and Ar is an optionally substituted aryl.]

2. The compound according to claim 1, wherein R¹ is —H, lower alkyl or benzyl; and Ar is an optionally substituted phenyl.

3. The compound according to claim, 1, wherein R¹ is —H or lower alkyl; R² is —H; and Ar is 4-fluoropheny.

4. A process for the production of the compound mentioned in claim 1 in which R² is —H comprising a reaction in which a dihydrooxazole derivative represented by the formula (II) 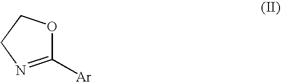
[In the formula, Ar is an optionally substituted aryl] with a nipecotic acid derivative represented by the formula (III) 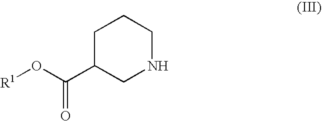
[In the formula, R¹ is —H or an ester residue] under an acidic condition and, when R¹ is a group other than —H, R¹ is removed if necessary.

5. A process for the production of the compound mentioned in claim 1 in which R¹ is —H or lower alkyl, R² is —H and Ar is 4-fluorophenyl comprising a reaction in which a compound represented by the formula (II) mentioned in claim 4 in which Ar is 4-fluorophenyl with a compound represented by the formula (III) mentioned in claim 4 in which R¹ is —H or lower alkyl under an acidic condition and, when R¹ is lower alkyl, R¹ is removed if necessary.

6. A process for the production of an isoquinoline derivative represented by the formula (IV) 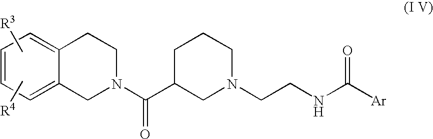
[In the formula, R³ and R⁴ are same or different and each is —H, lower alkyl or —O-lower alkyl; and Ar is an optionally substituted aryl], characterized in that, the compound mentioned in claim 1 is, if necessary, subjected to a reaction for removal of R¹ and/or R² in case R¹ and/or R² are/is groups other than —H, then condensed with a tetrahydroisoquinoline derivative represented by the formula (V) or a salt thereof 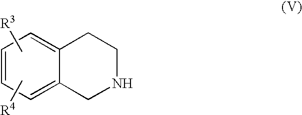
[In the formula, R³ and R⁴ are same or different and each is —H, lower alkyl or —O-lower alkyl] and, in case R² is a group other than —H, a reaction for removal of R² is conducted.

7. A process for the production of a compound represented by the formula (IV) mentioned in claim 6 in which R³ is 6-methoxy, R⁴ is 7-methoxy and Ar is 4-fluorophenyl, characterized in that, a compound mentioned in claim 1 in which R¹ is —H or lower alkyl, R² is —H and Ar is 4-fluorophenyl is subjected, if necessary, to a-reaction for removal of R¹ in case R¹ is lower alkyl and then condensed with a compound represented by the formula (V) mentioned in claim 6 in which R³ is 6-methoxy and R⁴ is 7-methoxy.
